(19) United States
(12) Patent Application Publication
(10) Pub. No.: US 2014/0018766 A1
(43) Pub. Date: Jan. 16, 2014

(54) APPARATUS AND METHOD FOR MOBILIZATION OF ENTRAINED GAS BUBBLES IN A FLUID CIRCUIT

(71) Applicant: Karen WHITE, Wellington, FL (US)

(72) Inventor: Karen WHITE, Wellington, FL (US)

(21) Appl. No.: 13/943,478

(22) Filed: Jul. 16, 2013

Related U.S. Application Data
(60) Provisional application No. 61/671,972, filed on Jul. 16, 2012.

(51) Int. Cl. A61M 1/36; A61M 5/14

(52) U.S. Cl. CPC A61M 1/3627; A61M 5/14
USPC 604/500; 96/175; 95/30

(57) ABSTRACT

An apparatus for mobilization of entrained gas bubbles in a fluid circuit comprises opposing fluid circuit engagement members. At least one of the fluid circuit engagement members has a vibrational energy transfer effector. A vibration generator is provided for generating vibrational energy. The vibrational energy transfer effector is coupled to the vibration generator such that vibrational energy is transmitted from the vibration generator to the vibrational energy transfer effector and to the fluid circuit. The vibration generator in one embodiment can produce vibrational oscillations at between 60 and 12000 cycles per minute. A method for mobilizing entrained gas bubbles in a fluid circuit is also disclosed.

### CROSS REFERENCE TO RELATED APPLICATIONS

This application claims the priority of U.S. provisional patent application Ser. No. 61/671,972 filed Jul. 16, 2012, the disclosure of which is incorporated fully herein.

### FIELD OF THE INVENTION

This invention relates generally to fluid circuit apparatus, and more particularly to fluid circuit apparatus for patient infusion and extracorporeal body fluids management.

### BACKGROUND OF THE INVENTION

The inadequate release and venting of air or other gases in medical procedures requiring fluid lines with blood circulation or delivery of therapeutics poses extreme risk to the patient. Air embolism may be fatal and upon occurrence frequently leads to stroke or debilitation of the patient. There are numerous adverse incidents traceable to procedural failures to remove air from fluid delivery circuits in the literature.

Typical medical procedures where air removal from a device or administration set containing blood or therapeutics is required, include without limitation oncology therapeutics and infusion, hemodialysis blood circulation, intravascular infusion therapy of drugs and agents, cardiopulmonary bypass support, cannulation of the heart for indefinite mechanical circulatory support, catheterization for therapeutics delivery, catheterization for diagnostic and radiological contrast media delivery, catheterization for the removal or exchange of blood or bypass circulation of blood during surgery, and ambulatory outpatient antibiotic therapy. Numerous other procedures where patent infusion or extracorporeal body fluids management tubes are required are also possible.

### SUMMARY OF THE INVENTION

An apparatus for mobilization of entrained gas bubbles in a fluid circuit comprises opposing fluid circuit engagement members. At least one of the fluid circuit engagement members has a vibrational energy transfer effector. A vibration generator is provided for generating vibrational energy. The vibrational energy transfer effector is coupled to the vibration generator such that vibrational energy is transmitted from the vibration generator to the vibrational energy transfer effector and to the fluid circuit. The vibration generator in one embodiment can produce vibrational oscillations at between 60 and 12000 cycles per minute.

The vibrational energy transfer effector can comprise a concave surface for engaging a cylindrical fluid circuit tube. The vibrational energy transfer effector can comprise a rotatable member. The rotatable member can be at least one selected from the group consisting of rollers, balls and cylinders. The vibrational energy transfer effector can comprise a platen. The vibrational energy transfer effector can comprise a low friction surface.

A vibration generator can be attached to each of the opposing fluid circuit engagement members, and each fluid circuit engagement member can comprise a vibrational energy transfer effector coupled to the respective vibration generator. One of the vibration generators in one embodiment can produce vibrational oscillations of less than 300 cycles per minute, and the opposing vibration generator can produce vibrational oscillations of greater than 6000 cycles per minute. One of the vibration generators can produce vibrational oscillations of between 60-300 cycles per minute, and the other vibration generator can produce vibrational oscillations of between 6000-12000 cycles per minute. One of the vibration generators can produce vibrational oscillations of 1-1000 cycles per second and the other vibration generator can produce ultrasonic vibrational oscillations of between 20,000-40,000 cycles per second.

The opposing fluid circuit engagement members can be provided as opposing jaws of pivotally connected scissor elements. The apparatus can further comprise at least one vibrational energy transfer effector protrusion element. The vibrational energy transfer effector protrusion element can be a tip element.

The apparatus can include a hand grip. The hand grip can comprise loops for at least the thumb and at least one of the opposing fingers. The hand grip can comprise a pistol grip and a thumb-actuated lever arm for causing one of the fluid circuit engagement members to move toward the opposing fluid circuit engagement member.

The opposing fluid circuit engagement members can be connected by a hinge. The opposing fluid circuit engagement members can be connected by a flexible material. The flexible material can include a wrist loop. The opposing fluid circuit engagement members can be connected by a wire form.

The apparatus can comprise at least one battery for powering the vibration generator. The apparatus can comprise at least one control element for controlling the operation of the vibration generator. The control element can be at least one selected from the group consisting of an on/off switch and a vibration oscillation speed control. The apparatus can further comprise a biasing member for biasing the opposing fluid circuit engagement members away from one another.

A method for mobilizing entrained gas bubbles in a fluid circuit including an elongated fluid circuit element comprises the step of contacting the elongated fluid circuit element with a vibrational energy transfer effector. The vibrational energy transfer effector imparts vibrational energy to the fluid circuit element. The vibrational energy transfer effector is moved along a length of the fluid circuit element while maintaining contact between the vibrational energy transfer effector and the fluid circuit element.

The method can comprise the step of generating vibrational energy in a vibration generator provided in at least one of opposing fluid circuit engagement members. The elongated fluid circuit element can comprise at least one selected from the group consisting of a patient infusion tube and an extracorporeal body fluids management tube. The opposing vibrational energy transfer effectors can be used to apply opposing vibrational energy to the elongated fluid circuit element.

One of the vibrational energy transfer effectors can impart to the elongated fluid circuit element vibrational oscillations of less than 300 cycles per minute, and the opposing vibrational energy transfer effector can impart to the elongated fluid circuit element vibrational oscillations of greater than 6000 cycles per minute. One of the vibrational energy transfer effectors can impart to the elongated fluid circuit element vibrational oscillations of between 60-300 cycles per minute, and the opposing vibrational energy transfer effector can impart to the elongated fluid circuit element vibrational oscillations of between 6000-12000 cycles per minute. One of the vibration energy transfer effectors can impart to the elongated fluid circuit element vibrational oscillations of 1-1000 cycles per second and the opposing vibrational energy transfer effector can impart to the elongated fluid circuit element ultrasonic vibrational oscillations of between 20,000-40,000 cycles per second.

### BRIEF DESCRIPTION OF THE DRAWINGS

There is shown in the drawings embodiments which are presently preferred, it being understood, however, that the invention can be embodied in other forms without departing from the spirit or essential attributes thereof, wherein:

FIG. 1 is a schematic diagram of an apparatus and method according to the invention.

FIG. 2 a-d are a schematic diagram of an apparatus according to the invention.

FIG. 3 is a perspective view of another embodiment, partially broken away to review internal features.

FIG. 4 is a perspective view of a third embodiment, partially broken away to reveal internal features.

FIG. 5 is a side elevation of a fourth embodiment, partially broken away to reveal internal features.

FIG. 6 is a side elevation of a fifth embodiment mounted to the hand and wrist of a user, partially in phantom.

FIG. 7 is a side elevation of a sixth embodiment, partially broken away to reveal internal features.

FIG. 8 is a side elevation of a seventh embodiment, partially broken away to reveal internal features.

FIG. 9 is a perspective view of an eighth embodiment, partially broken away to reveal internal features.

### DETAILED DESCRIPTION OF THE INVENTION

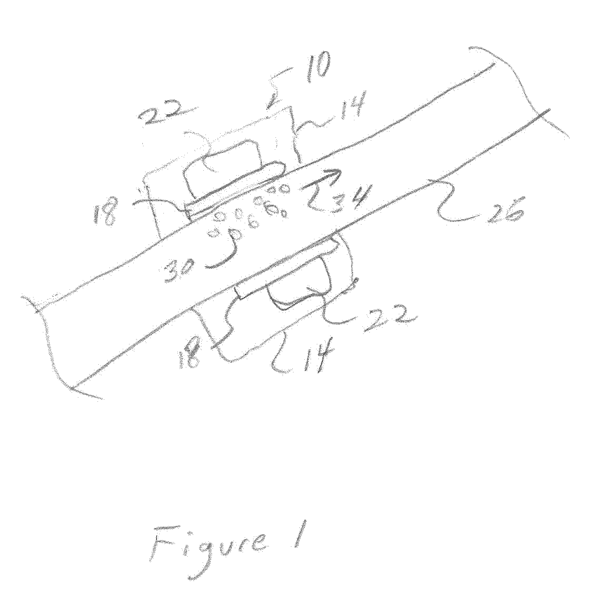

An apparatus for mobilization of entrained gas bubbles in a fluid circuit comprises opposing fluid circuit engagement members 14 as shown in FIG. 1. At least one of the fluid circuit engagement members 14 has a vibrational energy transfer effector 18. A vibration generator 22 is provided for generating vibrational energy. The vibrational energy transfer effector 18 is coupled to the vibration generator 22 such that vibrational energy is transmitted from the vibration generator 22 to the vibrational energy transfer effector 18 and to the fluid circuit element 26. The vibration generator in one embodiment can produce vibrational oscillations at between 60 and 12000 cycles per minute. The vibrational energy reaching the fluid circuit element 26 mobilizes gas bubbles 30 within the fluid circuit element 26 and causes the bubbles 30 to move in the direction of positive buoyancy as indicated by arrow 34.

The vibrational energy transfer effector 18 can comprise a concave surface for engaging a cylindrical fluid circuit tube. One vibrational energy transfer effector 18 can comprise a concave surface opposing an opposite vibrational energy transfer effector with a convex surface for engaging a cylindrical fluid circuit tube. The vibrational energy transfer effector 18 can comprise a rotatable member. The rotatable member can be at least one selected from the group consisting of rollers, balls and cylinders. The vibrational energy transfer effector can comprise a platen.

The vibrational energy transfer effector 18 can comprise a low friction surface. A low friction surface can be provided by any suitable means. Examples, without limitation, include hydrophilic and lubricious coatings, metallic anodizing treated surfaces, polymeric coatings such as polytetrafluoroethylene (PTFE) and Teflon®, highly polished surfaces, and surfaces treated with ceramic deposition.

A vibration generator 22 can be attached to each of the opposing fluid circuit engagement members 14, and each fluid circuit engagement member 14 can comprise a vibrational energy transfer effector 18 coupled to the respective vibration generator 22, as shown in FIG. 1. Any number of vibration energy transfer effectors are possible in any orientation about the fluid circuit element. Also, any number of vibration generators are possible. One of the vibration generators in one embodiment can produce vibrational oscillations of less than 300 cycles per minute, and the opposing vibration generator can produce vibrational oscillations of greater than 6000 cycles per minute. One of the vibration generators can produce vibrational oscillations of between 60-300 cycles per minute, and the other vibration generator can produce vibrational oscillations of between 6000-12000 cycles per minute. One of the vibration generators can produce vibrational oscillations of 1-1000 cycles per second and the other vibration generator can produce ultrasonic vibrational oscillations of between 20,000-40,000 cycles per second.

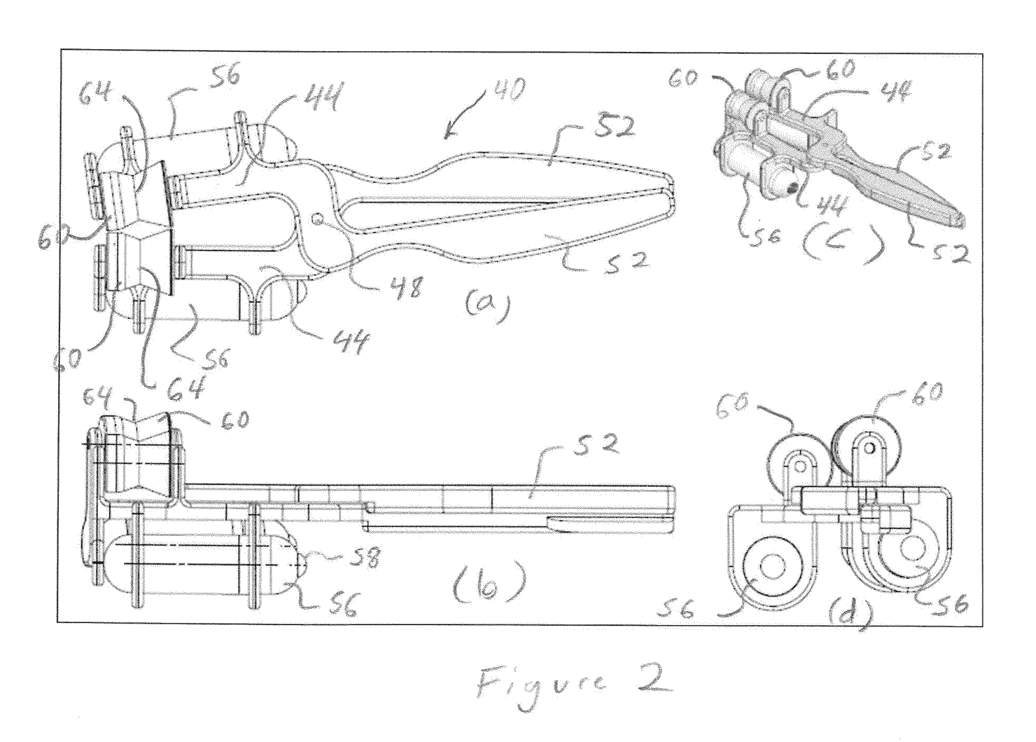

There is shown in FIG. 2 a first embodiment 40 in which opposing fluid circuit engagement members 44 are pivotally connected by pin 48 to handles 52 that are scissor or plier like elements. A vibration generator 56 can be provided on one or both of the fluid circuit engagement members 44. A pushbutton switch 58 can be used to power on and off the vibration generator 56. A vibrational energy transfer effector 60 is associated with each vibration generator 56 as by connection through a chassis such that vibrational energy is coupled through the vibrational energy transfer effector 60 to the fluid circuit element that is held between the fluid circuit engagement members 44. The vibrational energy transfer effectors 60 can have a concave portion 64 or other suitable structure for engaging the fluid circuit element. The vibration generator 56 can be a rotary vibrational motor and battery elements can be contained in a sealed motor housing. The scissor design allows opposing fluid circuit engagement members 44 with vibrational energy transfer effectors 60 to be clamped against tubing and fluid circuit elements by hand-finger control. The unit and rollers moved along the tubing in the fluid circuit disrupt immobilized adhered or pocketed gas or air bubbles.

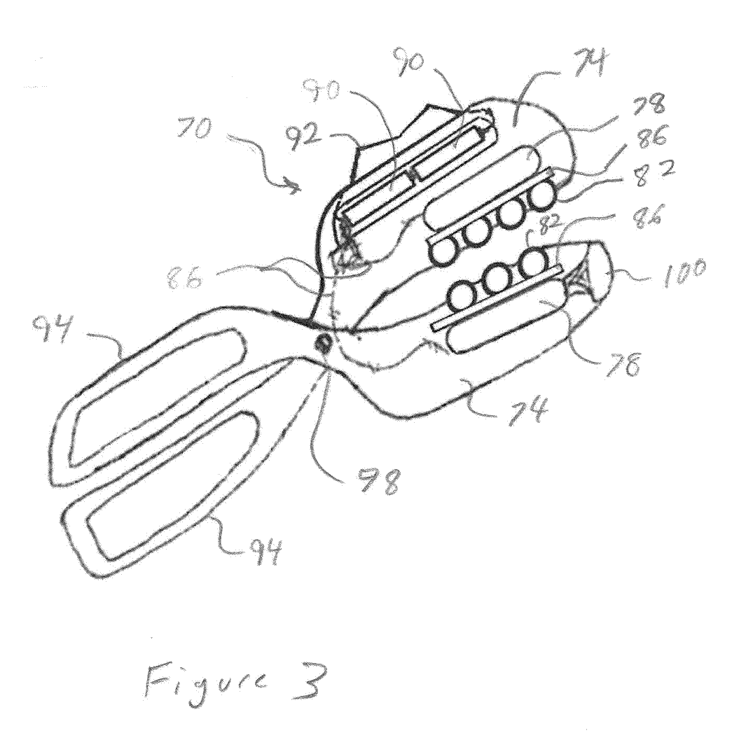

There is shown in FIG. 3 a second embodiment 70 having opposing fluid circuit engagement members 74. Vibration generators 78 are coupled through platens 86 to vibration energy transfer effectors 82 in the form of rotatable spherical elements or balls that are secured in the fluid circuit engagement members 74 by any suitable structure such as ganged along a raceway. The vibration generators 78 can receive power from batteries 90 through power lines 86. An on/off switch 92 can be provided. The opposing fluid circuit engagement members 74 are connected to scissor-like handles 94 with the grips in the form of loops for the thumb and at least one of the opposing fingers. The handles 94 can be pivotally connected at pivot pin 98. The apparatus can further comprise at least one vibrational energy transfer effector protrusion element 100. The vibrational energy transfer effector protrusion element can be a tip element. The tip 100 is in contact with the vibrational motor chassis and transmits vibrational energy through a solid mounted element to larger fluid circuit junctions, or in-line reservoirs and junction articles. This element allows for probing the junction or reservoir container external surfaces to release air adhered internally to the container or junction walls and surfaces. The protrusion tip element 100 is useful for applying vibrational energy to non-tubular fluid circuit elements which could not be readily positioned between the opposing fluid circuit engagement members 44.

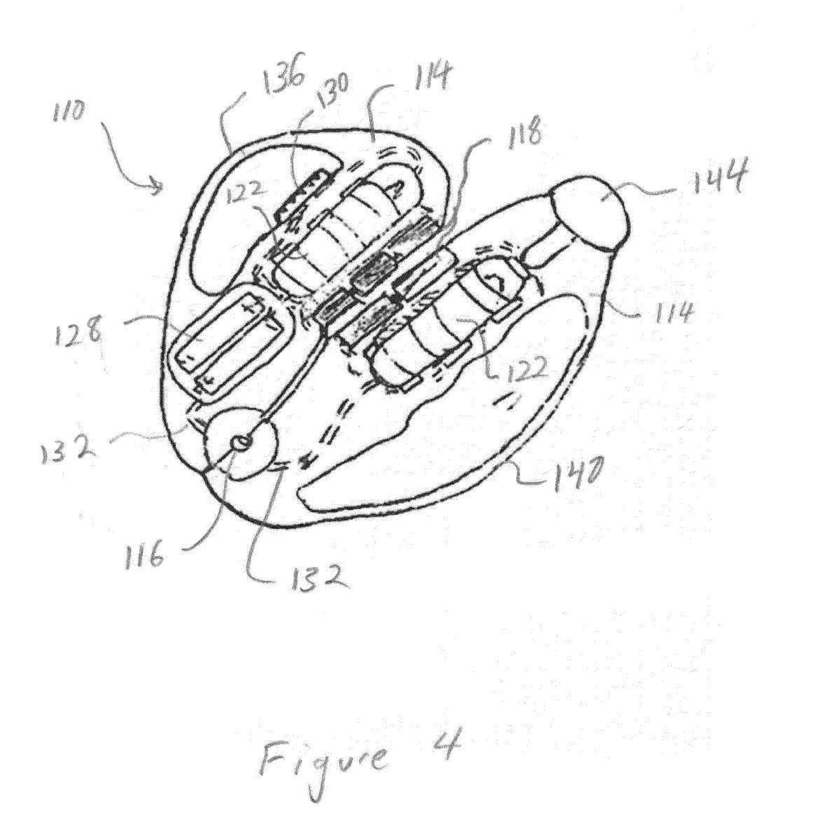

There is shown in FIG. 4 a third embodiment 110 of the apparatus. The apparatus 110 can have opposing fluid circuit engagement members 114 which can be hinged together by a hinge connection 116. Vibrational energy transfer effectors 118 can be coupled to vibration generators 122 such that vibrational energy is coupled to a fluid circuit element held between the opposing fluid circuit engagement members 114. Cylinder shaped metal beads or rollers are deployed as the transfer effectors 118 along a shaft, wire or race confinement and energized by contact with the vibration generators and supporting chassis elements. The roller beads are fixed in opposition in each fluid circuit engagement member 114. The fluid circuit element or tube is passed between the vibrational energy transfer effectors 118 with hand controlled pressure in the direction of air mobilization.

The vibration generators 122 can be powered by batteries 128 through power lines 132. An on/off switch 130 can be provided. The apparatus can include handgrips in the form of a loop 136 for the thumb and loop 140 for at least one of the opposing fingers. A vibration energy transfer effector protrusion element 144 can also be provided. The hinge element 116 allows for fluid circuit engagement member 114 opening and closing. The hinge element 116 may have a close pen spring shaped wire affixed about the pin or hinge screw (not illustrated), with each end of the wire form projecting to the inside of an opposing fluid circuit engagement member 114. In the manner described, the wire spring is formed to hold open the apparatus 110 when at rest with fluid circuit engagement member 114 angle of opening between approximately 20 and 40 degrees at rest. When the apparatus 110 is grasped by the user hand, closure is maintained on the fluid line by hand pressure.

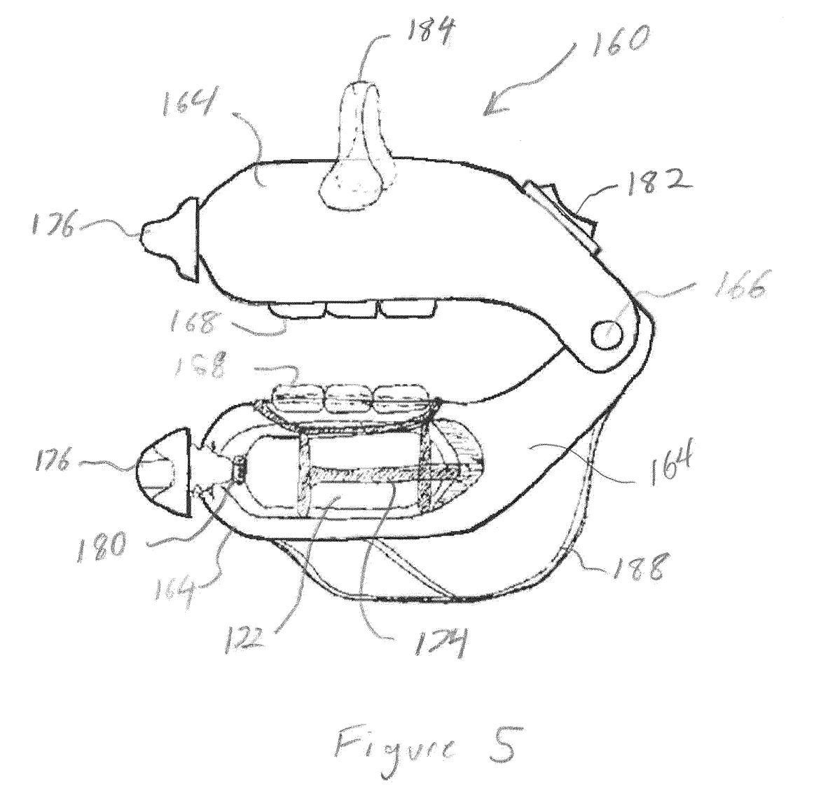

There is shown in FIG. 5 a fourth embodiment 160 of the apparatus. The apparatus 160 includes opposing fluid circuit engagement members 164. The opposing fluid circuit engagement members 164 can be hinged together as at hinge connection 166. Vibrational energy transfer effectors 168 in the form of elongated rollers or cylinders can be coupled to vibration generators 172 through chassis elements 174. The rollers 168 can be constructed of suitable materials such as metal or hard polymeric materials. Such elements 168 can be aligned on a shaft or wire and may also be held in a race against the vibration generators 172. The vibration generators 172 can include batteries (not shown) and a sealed motor on/off switch 180. A motor on/off screw 176 can be provided to engage switch 180 and a sliding switch 182 can also or alternatively be provided. Additionally the male thread element of each thumb screw 176 can be engaged in a female thread frame within the generator 172 mounting chassis. When the vibration generator 172 is on, the thumb screw 176 will also vibrate for probing objects in the target fluid circuit. A nut style on/off actuator may be designed with a push and turn to lock or release engagement to the chassis so as to prevent vibrational rotation of the nut during probing use. A hand grip in the form of a thumb loop 184 and loop 188 for at least one of the opposing fingers can be provided.

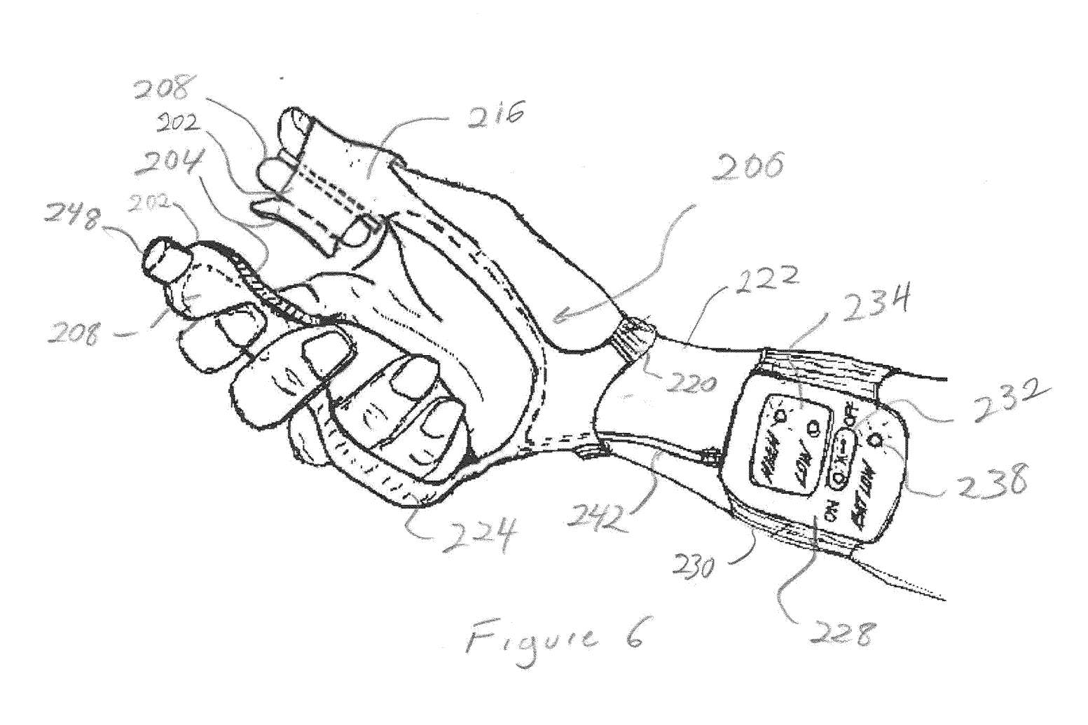

There is shown in FIG. 6 a fifth embodiment of the apparatus 200. The apparatus 200 includes opposing fluid circuit engagement members 202. The opposing fluid circuit engagement members 202 have vibrational energy transfer effectors 204 which can be coupled to vibration generators 208. A hand grip in the form of a thumb loop 216 and loop 224 for at least one of the opposing fingers can be provided. Additionally a wrist loop 220 can be provided to secure about the wrist of the user 222. The thumb loop 216, finger loop 224, and wrist loop 220 can all be constructed of a flexible material. The flexible material can be elastic such that the hand of the user 222 is securely engaged.

The apparatus 200 has a dual applicator design tethered to a power and control module 228 on a wrist strap 230. The top thumb loop 216 is used to hold one of the vibrational energy transfer effectors 204 to one side of the fluid circuit and the bottom applicator is secured by a rigid finger loop 224, and is used to hold the other of the vibrational energy transfer effectors 204 to the opposite side of the fluid circuit, thus compressing or gripping the desired circuit tubing or element to impart maximum opposing energy through the circuit. In like manner the device is moved along the fluid circuit to release and mobilize adhered or entrained air to the point of venting or egress as determined by the operator.

The ergonomic wrist secured design may have replaceable batteries in the wrist mounted module with on/off switch 232. A high or low vibrational energy switch 234 can be provided. A low battery indicator 238 can also be provided. A control line 242 can extend from the control module 228 to the vibration generators 208. Another version supplies the wrist band feature without power module, wherein the self-contained vibrator/battery motors are mounted in the separate applicator elements as previously described with on/off control at the motor housing end. The wrist band module 228 and other components can be provided in a single element. A vibrational energy transfer effector tip element 248 can also be provided.

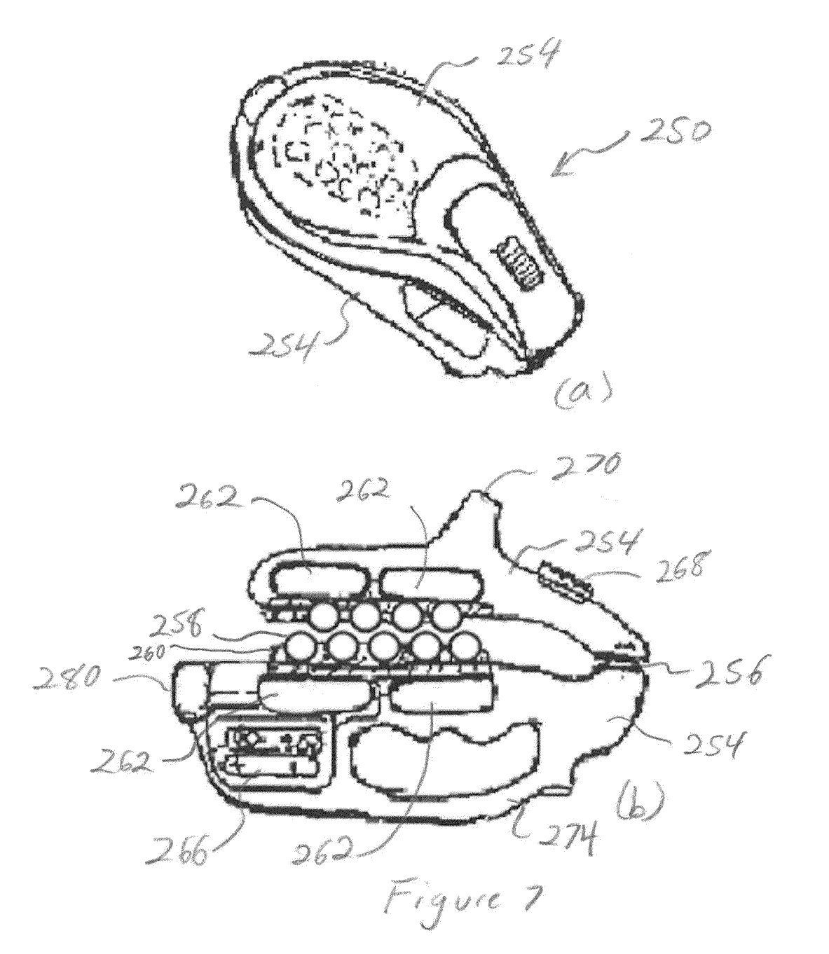

There is shown in FIG. 7 a sixth embodiment of the apparatus 250. The apparatus 250 includes opposing fluid circuit engagement members 254 which can be connected as by hinge element 256. Vibrational energy transfer effectors 258 can be provided in the form of a spherical elements or balls in a raceway 260. Raceway 260 may secure a two-dimensional array of energy transfer effectors to provide points of vibration transmission across a greater section of the fluid circuit element. Vibration generators 262 can be coupled to the vibrational energy transfer effectors 258 to impart vibrational energy to the vibrational energy transfer effectors 258 and thereby to a fluid circuit element. Batteries 266 can be mounted within the opposing fluid circuit engagement members 254 and can be switched on/off by switch 268. A vibrational energy transfer effector tip 280 can be provided and can be in contact with a vibration generator mounting chassis and receives vibrational energy for transfer to the targeted fluid circuit element. A hand grip in the form of a thumb loop 270 and a loop 274 for at least one of the opposing fingers can be provided.

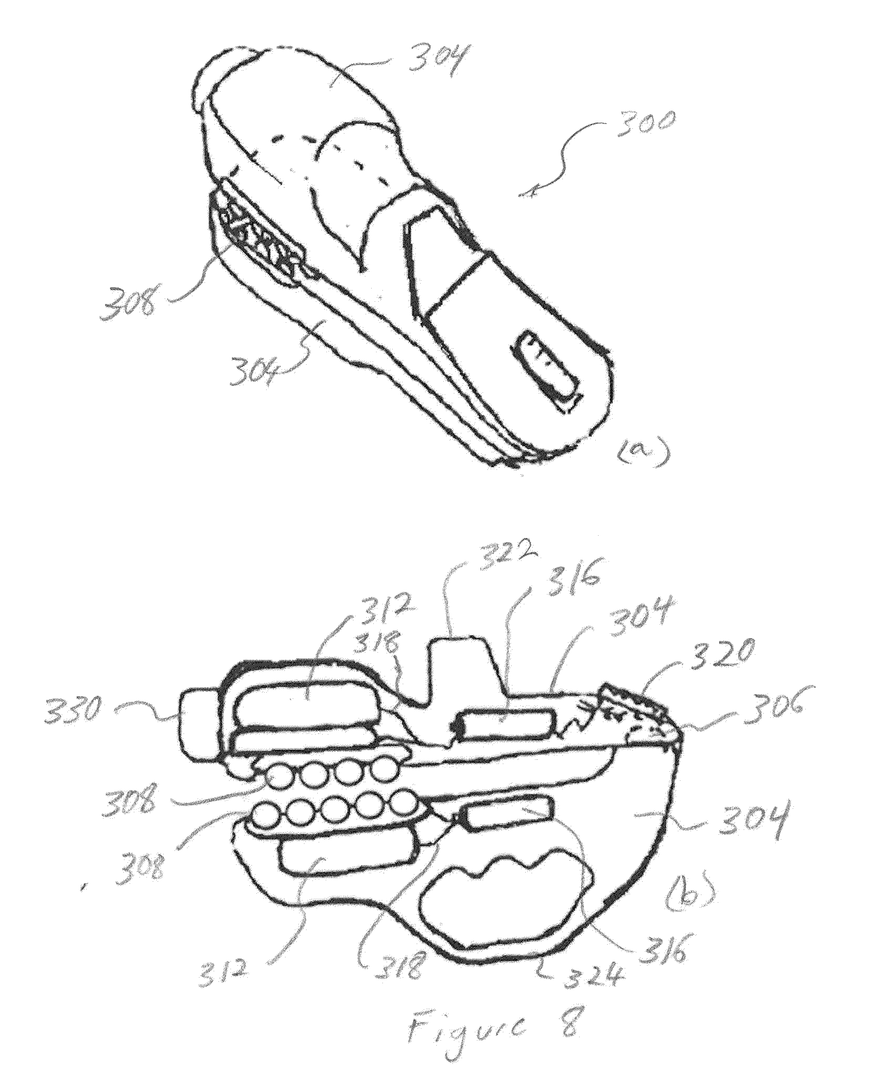

There is shown in FIG. 8 a seventh embodiment of the apparatus 300. The apparatus 300 includes opposing fluid circuit engagement members 304 having vibrational energy transfer effectors 308 and connected by hinge element 306. Vibration generators 312 are provided in each of the opposing fluid circuit engagement members 304. Batteries 316 are provided in supply power to the vibration generators 312 through power lines 318. A switch 320 can be provided. A hand grip in the form of a thumb loop 322 and a loop 324 for at least one of the opposing fingers can be provided. A vibrational energy transfer effector tip element 330 can be provided as previously described engaged with vibrational energy generator (not illustrated), in this embodiment on the fluid circuit engagement member 304 having the thumb loop 322 and constituting the top of the apparatus 300.

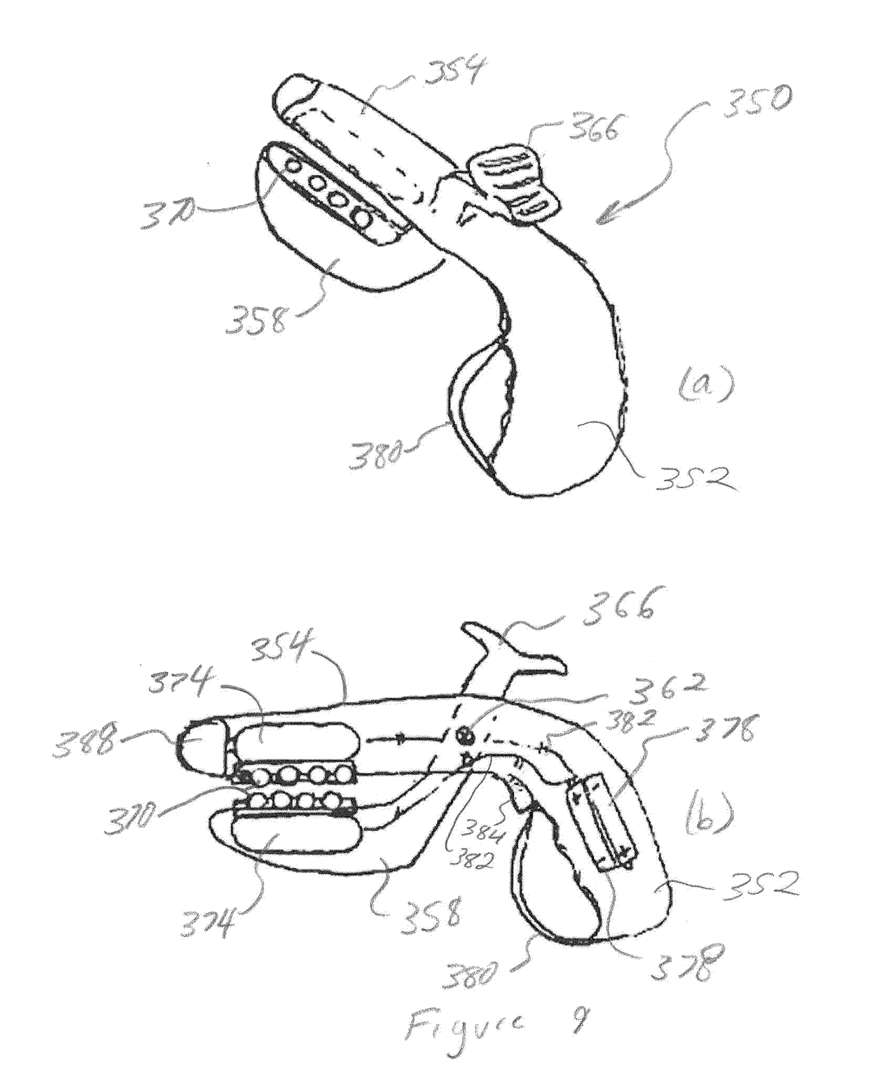

There is shown in FIG. 9 an eighth embodiment of the apparatus 350. The apparatus 350 includes a handle grip portion 352 having a first fluid circuit engagement member 354 connected thereto. The first fluid circuit engagement member 354 is contiguous to the pistol hand grip 352. A second opposing fluid circuit engagement member 358 is provided and pivotally engaged to the first fluid circuit engagement member 354 about a pivot connection 362. A thumb lever 366 can be provided to operate and move the second fluid circuit engagement member 358 relative to the first fluid circuit engagement member 354. Each fluid circuit engagement member has vibrational energy transfer effectors 370 and can have a vibration generator 374. Batteries 378 can be provided and supply power through power lines 382. A switch 384 in the form of the trigger can be provided. A finger loop 380 can be provided to ensure a secure grip.

A vibrational energy transfer effector tip protrusion 388 can also be provided. The vibrational energy transfer effector protrusion 388 can be a hard plastic element at the tip in contact with the upper vibration generator 374. The protrusion 388 transmits vibrational energy from the vibration generator 374 to fluid circuit elements.

A method for mobilizing entrained gas bubbles in a fluid circuit including an elongated fluid circuit element comprises the step of contacting the elongated fluid circuit element with a vibrational energy transfer effector. The vibrational energy transfer effector imparts vibrational energy to the fluid circuit element. The vibrational energy transfer effector is moved along a length of the fluid circuit element while maintaining contact between the vibrational energy transfer effector and the fluid circuit element.

The method can comprise the step of generating vibrational energy in a vibration generator provided in at least one of opposing fluid circuit engagement members. The elongated fluid circuit element can comprise at least one selected from the group consisting of a patient infusion tube and an extracorporeal body fluids management tube. Patient infusion can include, without limitation, transfusion and administration of blood products, Outpatient Antibiotic Therapy (OPAT) agents infusion, oncology therapeutics infusion, intravenous drug therapy administration and infusion, diagnostic and contrast media infusion for radiology, electrolyte replacement therapy, and peritoneal dialysis solution infusion. Extracorporeal body fluids management can include, without limitation, hemodialysis support, cardiopulmonary and bypass circulatory support, and cardioplegia and thermal transference to blood. Other uses are possible.

The opposing vibrational energy transfer effectors can be used to apply opposing vibrational energy to the elongated fluid circuit element. One of the vibrational energy transfer effectors can impart to the elongated fluid circuit element vibrational oscillations of less than 300 cycles per minute, and the opposing vibrational energy transfer effector can impart to the elongated fluid circuit element vibrational oscillations of greater than 6000 cycles per minute. One of the vibrational energy transfer effectors can impart to the elongated fluid circuit element vibrational oscillations of between 60-300 cycles per minute, and the opposing vibrational energy transfer effector can impart to the elongated fluid circuit element vibrational oscillations of between 6000-12000 cycles per minute. One of the vibration energy transfer effectors can impart to the elongated fluid circuit element vibrational oscillations of 1-1000 cycles per second and the opposing vibrational energy transfer effector can impart to the elongated fluid circuit element ultrasonic vibrational oscillations of between 20,000-40,000 cycles per second.

The vibration generators can be any suitable form of generator to produce oscillatory vibration cycles. Rotary generators and receptacle generators operating a drive shaft communicating with mechanical vibratory effectors is possible. Also solenoids and various electronic vibratory generators are possible.

Vibrational motors of rotary design typically rotate an offset weight on a shaft between 7500 to 12000 rpm; and create vibrational oscillations between 100 and 500 Hertz. A second low frequency impact generator, typically a linear slide element energized between electromagnetic poles within a housing, so as to reciprocate along the axis of the alternating poles and create a bimodal impact in the range of 60-300 Hertz may be ganged with vibrator motors or mounted separately. The low frequency impact cycles imparts and transfers resonating impact pulses of energy through energy transfer elements of the apparatus in contact with target fluid or blood line circuit components.

Alternatively, a hand held device powered by DC current from batteries, with the apparatus fashioned in a hand gripped device with opposing applicators. The vibrational energy transfer effectors contain rollers, bearing balls, or cylinders of hard material constrained by the applicator structure. These elements are in contact with the vibrational and/or bimodal impact components of the apparatus. When the fluid circuit engagement members of the apparatus are closed around a tube, such as a perfusion line or blood line, the device is moved easily down the line in the direction of desired air mobilization. The operator may control by one hand the force of fluid circuit engagement member or vibrational energy transfer effector closure in contact with the line to contact or compress the fluid circuit element and impart energy through the vibrational energy transfer effectors to disassociate and mobilize entrained or trapped air in the fluid line. The operator uses one hand to manage the fluid line while pulling the hand held device in the other hand along the tubing in the direction or air venting. This provides progression of the device along the fluid line to mobilize trapped air to the desired air venting or removal point in the fluid element circuit in a controlled and incremental fashion.

A further feature of the device is separate frequency modulation of the vibrational elements. By combining one frequency in one fluid circuit engagement member and another frequency in the other fluid circuit engagement member of the apparatus; the impact on static air immobilized by surface tension on internal fluid surfaces in a tubing or fluid line may be enhanced. Entrained air and air bubbles adhered by surface tension to surfaces are energized to release and mobilize in the fluid circuit in the direction of apparatus application along the circuit in the preferred buoyancy direction. Typically the fluid circuit is positioned to allow buoyancy of air as mobilized to migrate to a preferred point of venting from the circuit.

Vibration motors acting on electromagnetic or piezoelectric coupling to metal or ceramic motor slides or oscillating elements provide displacement energy to vibrational energy transfer effectors. The vibration generators may be cylindrical or disk shaped. Cylindrical motors useful in the device design typically measure 10-25 mm in length and 4-12 mm in diameter. More than one motor may be ganged in the design, and different vibrational frequency motors may be ganged or opposed in different fluid circuit engagement members to impart more than one frequency of vibration to the vibrational energy transfer effector elements of the design, and thus the targeted fluid circuit contact points. Typical vibrational frequencies of motors for the preferred design are in the range of 6,000 to 12,000 impacts per minute for rotary mechanism vibrator designs. Voltage requirements range from 1 to 3 volts for each motor typically in a range of 60-120 milliamps current. Vibrator motors may have contacts or lead wires exposed for battery connection, or may have fully enclosed batteries sealed within the motor housing. Vibrational motors may be fully enclosed in metal or plastic encasements which are then mounted within the fluid circuitjaw enclosure design and making contact with the vibrational energy transfer effector elements of the design which make contact with the targeted fluid circuit elements containing entrained air bubbles.

A biomodal impact generator mechanism in which a weighted cylinder or ball slides along a shaft or is enclosed in a race/chassis frame and when energized by electromagnetic or piezoelectric coupling provides a hammer impact energy at low frequency, typically 60-300 impacts per minute. The impact hammer element may work in tandem with the higher frequency vibrator motors, usually in the opposite applicator jaw element.

Singularly or in tandem the vibration motors and impact hammer mechanisms transmit disruptive force through applicator elements to air bubbles adhered to internal fluid circuit tubing and connectors. Thus, air bubbles are disruptively agitated from stiction or surface tension adhesion on tubing or connector surfaces. Released air bubbles and soluble air in the fluid are mobilized and aided into the fluid media by vibration pulses and forced along the fluid pathway in the direction of device application along the line toward vent or egress locations in the circuit by buoyancy control of the line or fluid pathway.

The vibration generators are housed in the device fluid circuit engagement members and make contact with vibrational energy transfer effectors made of ferrous and non-ferrous metallic rollers or balls or hard polymer substrates machined to the preferred shape for contact with the tubing or fluid circuit. The vibrational energy transfer effectors are retained by suspension design in the fluid circuit engagement member structure such as a cage or bracket element so as to absorb and transmit vibrational and hammer impact energy from the bimodal impact and motor generated vibrational element(s). Typical vibrating effector elements are ganged side by side in the suspension retainer or chassis in an array of 2-12 elements and interface independently with a common vibrational motor contact element. Spherical metal or hard plastic elements are typically 5-8 mm in diameter. Roller elements are typically 10-20 mm in length and 5-8 mm in diameter. The suspension allows for the effector elements to vibrate and roll along the surface of the tubing when in opposing jaw construct as applied in a scissor design to tubing. Such application elements transfer disruptive energy to the targeted fluid circuit elements, without degradation of the fluid circuit components, device suspension retainer elements, or device housing structure.

The device housing and opposing fluid circuit engagement members are sealed as necessary to prevent moisture and fluid intrusion to the electrical elements of the design. The device housing and fluid circuit engagement members may be made of thermoplastic materials so as to be molded, or fabricated from cast metal, machined metal such as stainless steel, or liquid metal injection forming to provide the desired ergonomic shape of the device for single handed use. Fabrication may include injection or deposition of adhesives, silicone or epoxies for joining and sealing design elements.

A switch to enable power to vibrational motors is mounted in the housing or along the ridge of one fluid circuit engagement member for thumb actuation, or in another suitable location. The switch is located so as to prevent inadvertent power switching when the device is gripped for application. In at least one embodiment of the design a single battery compartment is included in the fluid circuit engagement member or handle and provides power through the thumb switch to two vibrator motors, one in each fluid circuit engagement member. In a separate embodiment of the design, each of the vibration generators has a depression switch and battery housed within the motor casing on one end and sealed so as to be exposed in the fluid circuit engagement members for switch depression by the operator. Such motors typically provide approximately 100 hours of continuous operation before replacement is required. Vibrator motors with included switch and battery are easily exchanged within the frame or housing of the jaw element in some embodiments.

The apparatus may be injection molded from thermoplastics with compartments for mounting motors, switches, on/off screws and strap attachments. Alternatively, a framework of wire, metal stampings or plastic molded structure may be formed to receive the motors, applicator elements and race, screws and straps. The assembled substructure may be over formed by heat shrinkable plastics or dip coated rubbers or by sliding rubber or plastic forms over the substructure to finish the jaw element.

The device may also be applied to numerous other fluid delivery line applications where entrained air requires release and venting including; sample injection lines for liquid chromatography, precise fluid metering circuits, anaerobic mixing lines and other fluid line preparation procedures without limitation.

The apparatus vibration generator can be provided with the vibrational energy transfer effector as a single unit. The vibration generator can be coupled to at least one of the fluid circuit engagement members and the vibration generator can comprise a vibrational energy transfer effector in direct contact with the fluid circuit elements. The vibration generator can be sheathed or coated with a low friction surface to form a vibrational energy transfer effector that is integral with the vibration generator. The vibrational energy transfer effector can be molded or formed with or as part of a vibration generator housing. Other constructions are possible.

This invention can be embodied in other forms without departing from the spirit or essential attributes thereof, and accordingly, reference should be had to the following claims to determine the scope of the invention.

What is claimed is:

1. An apparatus for mobilization of entrained gas bubbles in a fluid circuit, comprising opposing fluid circuit engagement members, at least one of the fluid circuit engagement members having a vibrational energy transfer effector, and a vibration generator for generating vibrational energy, the vibrational energy transfer effector being coupled to the vibration generator such that vibrational energy is transmitted from the vibration generator to the vibrational energy transfer effector and to the fluid circuit.

2. The apparatus of claim 1, wherein the vibration generator produces vibrational oscillations at between 60 and 12000 cycles per minute.

3. The apparatus of claim 1, wherein the vibrational energy transfer effector comprises a concave surface for engaging a cylindrical fluid circuit tube.

4. The apparatus of claim 1, wherein the vibrational energy transfer effector comprises a rotatable member.

5. The apparatus of claim 4, wherein the rotatable member is at least one selected from the group consisting of rollers, balls and cylinders.

6. The apparatus of claim 1, wherein the vibrational energy transfer effector comprises a platen.

7. The apparatus of claim 1, wherein the vibrational energy transfer effector comprises a low friction surface.

8. The apparatus of claim 1, wherein a vibration generator is attached to each of the opposing fluid circuit engagement members, and each fluid circuit engagement member comprises a vibrational energy transfer effector coupled to the respective vibration generator.

9. The apparatus of claim 8, wherein one of the vibration generators produces vibrational oscillations of less than 300 cycles per minute, and the opposing vibration generator produces vibrational oscillations of greater than 6000 cycles per minute.

10. The apparatus of claim 8, wherein one of the vibration generators produces vibrational oscillations of between 60-300 cycles per minute, and the other vibration generator produces vibrational oscillations of between 6000-12000 cycles per minute.

11. The apparatus of claim 8, wherein one of the vibration generators produces vibrational oscillations of between 1-1000 cycles per second, and the other vibration generator produces vibrational oscillations of between 20000-40000 cycles per second.

12. The apparatus of claim 1, wherein the opposing fluid circuit engagement members are provided as opposing jaws of pivotally connected scissor elements.

13. The apparatus of claim 1, further comprising at least one vibrational energy transfer effector protrusion element.

14. The apparatus of claim 13, wherein the vibrational energy transfer effector protrusion element is a tip element

15. The apparatus of claim 1, further comprising a hand grip.

16. The apparatus of claim 15, wherein the hand grip comprises loops for at least the thumb and at least one of the opposing fingers.

17. The apparatus of claim 15, wherein the hand grip comprises a pistol grip and a thumb-actuated lever arm for causing one of the fluid circuit engagement members to move toward the opposing fluid circuit engagement member.

18. The apparatus of claim 1, wherein the opposing fluid circuit engagement members are pivotally connected.

19. The apparatus of claim 1, wherein the opposing fluid circuit engagement members are connected by a flexible material.

20. The apparatus of claim 19, wherein the flexible material includes a wrist loop.

21. The apparatus of claim 1, further comprising at least one battery for powering the vibration generator.

22. The apparatus of claim 1, further comprising at least one control element for controlling the operation of the vibration generator.

23. The apparatus of claim 22, wherein the control element is at least one selected from the group consisting of an on/off switch and a vibration oscillation speed control.

24. The apparatus of claim 1, further comprising a biasing member for biasing the opposing fluid circuit engagement members away from one another.

25. The apparatus of claim 1, wherein the vibration generator and the vibrational energy transfer effector are provided as a single unit.

26. A method for mobilizing entrained gas bubbles in a fluid circuit comprising an elongated fluid circuit element, comprising the steps of contacting the elongated fluid circuit element with a vibrational energy transfer effector, the vibrational energy transfer effector imparting vibrational energy to the fluid circuit element, and moving the vibrational energy transfer effector along a length of the fluid circuit element while maintaining contact between the vibrational energy transfer effector and the fluid circuit element.

27. The method of claim 26, further comprising the step of generating vibrational energy in a vibration generator provided in at least one of opposing fluid circuit engagement members.

28. The method of claim 26, wherein the elongated fluid circuit element comprises at least one selected from the group consisting of a patient infusion tube and an extracorporeal body fluids management tube.

29. The method of claim 26, wherein opposing vibrational energy transfer effectors are used to apply opposing vibrational energy to the elongated fluid circuit element.

30. The method of claim 29, wherein one of the vibrational energy transfer effectors imparts to the elongated fluid circuit element vibrational oscillations of less than 300 cycles per minute, and the opposing vibrational energy transfer effector imparts to the elongated fluid circuit element vibrational oscillations of greater than 6000 cycles per minute.

31. The method of claim 29, wherein one of the vibrational energy transfer effectors imparts to the elongated fluid circuit element vibrational oscillations of between 60-300 cycles per minute, and the opposing vibrational energy transfer effector imparts to the elongated fluid circuit element vibrational oscillations of between 6000-12000 cycles per minute.

32. The method of claim 29, wherein one of the vibrational energy transfer effectors imparts to the elongated fluid circuit element vibrational oscillations of between 1-1000 cycles per second, and the opposing vibrational energy transfer effector imparts to the elongated fluid circuit element vibrational oscillations of between 20000-40000 cycles per second.
